(19) United States
(12) Patent Application Publication
(10) Pub. No.: US 2026/0240129 A1
(43) Pub. Date: Aug. 20, 2026

(54) METHOD FOR CULTIVATION OF BLACK SOLDIER FLY LARVAE USING HIGH-CONCENTRATION ORGANIC WASTEWATER AND TRANS-ABDOMINAL PREDIGESTION OF PLANT-BASED FEEDSTUFF BY BLACK SOLDIER FLY LARVAE

(71) Applicant: Bioforte Biotechnology (Shenzhen) Co., Ltd., Shenzhen (CN)

(72) Inventors: Wenfeng HU, Shenzhen (CN); Jianfeng ZHU, Shenzhen (CN); Ruiting GUO, Shenzhen (CN); Jiaojiao QI, Shenzhen (CN); Chong MA, Shenzhen (CN)

(73) Assignee: Bioforte Biotechnology (Shenzhen) Co., Ltd., Shenzhen (CN)

(21) Appl. No.: 19/529,202

(22) Filed: Feb. 4, 2026

(30) Foreign Application Priority Data
Feb. 18, 2025 (CN) 2025101759050

(51) Int. Cl. A01K 67/368; A23K 10/12; A23K 20/105; A23K 20/142; A23K 20/28; A23K 50/90

(52) U.S. Cl. CPC A01K 67/368; A23K 10/12; A23K 20/105; A23K 20/142; A23K 20/28; A23K 50/90; A01K 2227/706

(57) ABSTRACT

The present disclosure belongs to the technical field of organic wastewater resource utilization, and discloses a method for cultivation of black soldier fly larvae using high-concentration organic wastewater and trans-abdominal predigestion of plant-based feedstuff by black soldier fly larvae. The method comprises: collecting organic wastewater into a feed, inoculating a fermentation agent for fermentation, adding modified zeolite, folic acid, and choline chloride into the fermentation product to be stirred uniformly, and adding the organic wastewater to adjust a humidity of a mixture to obtain the feed; and feeding the black soldier fly larvae with the resulting feed, and adding the organic wastewater to maintain a humidity of the feed in a feeding process. According to the present disclosure, pre-treatment of the organic wastewater is not required, multiple use in feed formulation and feeding is achieved.

### CROSS-REFERENCE TO RELATED APPLICATIONS

The application claims priority to Chinese patent application No. 2025101759050, filed on Feb. 18, 2025, the entire contents of which are incorporated herein by reference.

### TECHNICAL FIELD

The present disclosure belongs to the technical field of organic wastewater resource utilization, and particularly relates to a method for cultivation of black soldier fly larvae (BSFL) using high-concentration organic wastewater and trans-abdominal predigestion of plant-based feedstuff by BSFL.

### BACKGROUND

Organic wastewater generally refers to wastewater having a COD of 2000 mg/L or more discharged from paper-making, leather, food processing and other industries. These kinds of wastewater contain a large quantity of organic matters such as carbohydrates, fats, proteins, and celluloses, which cause serious pollution if discharged directly. Meal waste accounts for about 30%-50% of solid waste worldwide, and meal wastewater produced by separation from meal waste has a high BOD₅ concentration as high as 50,000 mg/L or more and has complex compositions, mainly consisting of a large quantity of soluble organic matters and suspended matters and having rich sugars, fats, proteins, amino acids, organic acids, and various trace elements. It is both a valuable resource and a source of pollution; and it is easily spoiled, rancidified, and malodorous due to extremely high moisture, organic matters and a large quantity of microorganisms, but can be converted into a new resource after proper treatment and processing. Fats are a major contributor to a COD/BOD₅ value in meal wastewater, and because of its content of fatty acids, glycerol, and other ingredients, they are not easily biodegradable and severely hinder subsequent biological treatment processes. In addition, high-concentration wastewater may also be produced in other food processing processes, such as wastewater produced in dairy plants, fruit processing plants, bean product plants, brewhouses, starch saccharification plants, amino acid fermentation plants, organic acid fermentation plants, and the like. In general, organic wastewater having a high COD/BOD₅ should be specially treated, the process is complicated, the investment is large, and the operating cost is high.

Currently, meal waste treatment is mainly directed to waste residues in a relatively large quantity of ways, such as landfill, incineration, aerobic composting, anaerobic fermentation, insect bioconversion, and the like. Because the COD and BOD₅ of the organic wastewater can be as high as tens or hundreds of thousands, the organic wastewater is currently treated anaerobically and aerobically to produce biogas, and the treatment cost is high. For example, conventional disposal of meal wastewater is often subjected to residue-oil-water three-phase separation to be treated or discharged as general domestic sewage, which not only increases the disposal cost of meal wastewater, but also wastes utilization of nutrients in meal wastewater. Biological treatment methods disclosed in CN118558707A and CN117179126A generally employ the manner of filtering off wastewater and only adding waste residues into insect feeds, which tends to cause waste of organic matters in the wastewater. However, the separated meal wastewater is high-concentration organic wastewater, the COD can be as high as 100 thousand (mg/L) or more, indicating that the water contains a large quantity of organic pollutants, which may include toxic and harmful organic compounds, have toxic effects on aquatic organisms, destroy aquatic ecological balance, and pose a threat to human health and drinking water. When wastewater containing organic matters is used directly for feeding livestock and poultry or fish, the high-concentration COD results in decreased feed intake, the harmful substances contained therein destroy their immunity, and nutrients of the wastewater itself alone are not sufficient to satisfy nutrient requirements of animals, resulting in generally lower daily weight gains and survival rates.

The role of resource insects, such as edible insects, in a treatment process of waste containing organic matters has become a hot spot of research, and after degradation by trans-abdominal digestion of insects, the organic matters can be converted into high-value-added products, enabling efficient use of resource cycles. Therefore, how to reasonably recycle high-concentration COD/BOD₅ organic wastewater, bio-convert it into resource-based substances, reduce treatment costs and increase economic conversion values, achieve resource utilization of organic wastewater, and improve ecological environment is important.

### SUMMARY

In order to solve the shortcomings of the prior art, one of objects of the present disclosure is to provide a method for cultivation of BSFL using high-concentration organic wastewater and trans-abdominal predigestion of plant-based feedstuff by BSFL.

Another object of the present disclosure is to provide a black soldier fly feed prepared based on high-concentration organic wastewater.

To achieve the above objects, the present disclosure provides the following technical solutions:

A method for cultivation of BSFL using high-concentration organic wastewater, including the following steps:

S1. collecting the high-concentration organic wastewater, adding the organic wastewater into a plant-based feed to achieve a moisture content of 60% or above, and inoculating a fermentation agent for fermentation treatment to obtain a fermentation product; then, adding modified zeolite, folic acid, and choline chloride into the fermentation product, and stirring uniformly to obtain a feed, where the organic wastewater added not only regulates a humidity of a mixture, but also is rich in organic nutrients; and

S2. feeding the BSFL with the feed obtained in step S1, and maintaining a humidity of the feed by directly supplementing the organic wastewater of step S1 according to a water evaporation condition and content in a feeding process.

Further, a COD or BOD₅ value of the high-concentration organic wastewater in step S1 is greater than 50,000 mg/L.

Further, the organic wastewater in step S1 is obtained from meal three-phase separation wastewater, and may also be high-concentration wastewater produced in other food processing processes, such as wastewater produced in dairy plants, fruit processing plants, bean product plants, starch saccharification plants, brewhouses, amino acid and organic acid fermentation plants, and the like.

Further, an amount of the high-concentration organic wastewater added in step S1 is to achieve a water content of the plant-based feed of 65%-85%, and meanwhile, abundant nutrients, including sugars, fats, proteins, amino acids, and the like are supplemented.

Further, the plant-based feed in step S1 can be selected from soybean meal, rapeseed meal, cottonseed meal, peanut meal, sunflower meal, palm kernel meal, coconut meal, dry and wet distillers' grains, DDGS, Chinese medicine residues, and other plant-based raw materials rich in nutrients such as fibers, sugars, starches, fats, and proteins.

According to the present disclosure, a plant-based feed having a high fiber content is used as a carrier, not only the plant-based feed contains sugars, starches, proteins, and fats, but also the fiber in the fermentation and black soldier fly cultivation process generates a large amount of fermentation heat to evaporate wastewater, so the cultivation process also needs the supplementation of organic wastewater to achieve the purposes of concentrating and evaporating water, thereby realizing reduction of wastewater; and the concentrated wastewater contains abundant sugars, starches, proteins, and fats, and provides excellent nutrients for the growth and development of BSFL.

According to the present disclosure, high-concentration organic wastewater is directly utilized as one of nutritional ingredients, is mixed with the plant-based feed described above, and is directly used for the cultivation of BSFL after several days of fermentation, thereby creating a brand-new recovery treatment method of organic high-concentration wastewater; and each black soldier fly larva is a biochemical reactor, and after the organic wastewater is degraded through trans-abdominal digestion by insects, it can be converted into high-value-added products, realizing efficient use of waste resource recycling.

Further, the modified zeolite in step S1 is obtained by a modification method of reducing a silica-alumina ratio, KCl and NaOH are dissolved in water, Al(OH)₃ is dissolved in a hot KCl and NaOH solution, natural zeolite is added into the solution and stirred at a normal temperature for 3-5 h, the solution is separated, and water is used for washing until neutral. After evaporation, the solid is calcined at 350-500° C. for 1.5-2 h. In the study of the present disclosure, the modified zeolite can improve the survival rate of the BSFL, improve feed intake, and promote nutrient absorption conversion; and it is possible that the modified zeolite changes the adsorption characteristics and adsorption effect of the zeolite compared to natural zeolite.

Further, the fermentation agent in step S1 is one or a mixture of more of yeast, Bacillus subtilis, and lactic acid bacteria.

Further, a using amount of the fermentation agent in step S1 is 0.5%-0.8% of a mass of the plant-based feed.

Further, in step S1, a fermentation temperature is 28-35° C., and a fermentation time is 1-5 days, which are adjusted according to a temperature and a pH value of raw materials.

Further, the feed in step S1 includes 500-700 parts of fermentation product, 14-28 parts of modified zeolite, 0.00005-0.0003 parts of folic acid, and 0.12-0.16 parts of choline chloride, and the organic wastewater is added to adjust the humidity of the feed at 60%-80%.

Further, in step S2, the black soldier fly larvae are selected to be 5-6 days old.

Further, the humidity of the feed in step S2 is maintained at 60%-80%.

The present disclosure also provides a black soldier fly larva feed prepared by adopting the above method based on recycling of high-concentration organic wastewater. The components of the feed include a plant-based feed, organic wastewater, a fermentation agent, modified zeolites, folic acid, and choline chloride.

The present disclosure also provides use of the above method for feeding BSFL based on high-concentration organic wastewater in improving growth performance of BSFL, and the growth performance includes weight gains, survival rates, feed conversion ratios, and the like. In addition, it has been found through studies that feeding with the black soldier fly feed prepared according to the present disclosure can also increase the fatty acid content, particularly the lauric acid content in black soldier fly bodies.

Further, the present disclosure further claims insect litter after trans-abdominal treatment of black soldier flies collected after feeding in the above step S2. The above insect litter mainly refers to BSFL exuviae after trans-abdominal treatment of black soldier flies, residues after molting of larvae, insect excretions in the feeding process, and other matters. The resulting insect litter is used for farming livestock and poultry, and can improve adverse effects in the livestock and poultry farming process, such as the production performance and death and culling rate, due to the use of miscellaneous meal type raw materials.

Compared with the prior art, the present disclosure has the following beneficial effects:

According to the present disclosure, pre-treatment of organic wastewater with a high-concentration COD/BOD₅ is not required, multiple use in feed formulation is achieved, direct addition for feeding BSFL can be achieved, resource recycling of organic wastewater with the high-concentration COD/BOD₅ is realized, the ecological environment is improved, and the process is simple.

According to the present disclosure, the BSFL are fed with the feed prepared by adding the modified zeolite, so that the feed intake conversion and tolerance of the BSFL to the feed based on organic wastewater with the high-concentration COD/BOD₅ can be improved, and the growth performance, including the survival rates, weight gains, and feed conversion ratios, etc., of the BSFL can be significantly improved.

According to the present disclosure, adding folic acid and choline chloride enhances both the growth performance of BSFL and their fatty acid content-particularly lauric acid content. The resulting high-protein, high-organic-matter insect litter can be utilized to produce chicken feed, swine feed, aquatic feed, and herbivore feed, enabling the direct conversion of the litter into feedstock. This not only delivers enhanced economic value but also accomplishes the transformation of low-value wastewater into high-value products.

### DETAILED DESCRIPTION OF THE EMBODIMENTS

The following are the specific examples of the present disclosure, which will provide a detailed and comprehensive explanation of the technical solution of the present disclosure. It is to be noted that the examples provided represent only a part, not all, of the present disclosure. Based on the examples in the present disclosure, all other examples acquired by those of ordinary skill in the art without making creative efforts shall fall within the scope of protection of the present disclosure.

Test methods used in the examples of the present disclosure are conventional methods unless otherwise specified; and materials, reagents, and the like used are commercially available reagents and materials unless otherwise specified.

Zeolite is modified using a conventional method: KCl and NaOH are dissolved in water, Al(OH) 3 is dissolved in a hot KCl and NaOH solution (a molar ratio of 1:1:1), natural zeolite is added into the solution and stirred at a normal temperature for 3 h, the solution is separated, and water is used for washing until neutral. After evaporation, the solid is calcined at 500° C. for 1.5 h.

The choline chloride content (%) is 60%, and is purchased from Wuhan Jiyesheng Chemical Co., Ltd.; the auxiliary bacterial agents are purchased from the China Center of Industrial Culture Collection, with the Chinese name Yarrowia lipolytica, and the No. CICC31829; purchased from the China Center of Industrial Culture Collection, with the Chinese name Bacillus subtilis, and the No. CICC10446; and purchased from the China Center of Industrial Culture Collection, with the Chinese name Lactobacillus acidophilus, and the No. CICC6082. The above-mentioned bacterial agents are not limited to the above-mentioned numbers, and other sources of said bacterial agents can be used by those skilled in the art.

In some of the examples and the comparative examples of the present disclosure, the organic wastewater adopts meal wastewater from a kitchen waste disposal station in Baiyun District, Guangzhou City, Guangdong Province. The examples and the comparative examples of the present disclosure adopt the same batch of meal waste for optimization testing; after removing impurities such as plastic and metal, the waste is pulverized by a pulverizer, and then meal wastewater is collected by separation using a three-phase separation device. It is tested that the COD of the meal wastewater is 154,620 mg/L. The organic wastewater in some examples (e.g., Example 5) adopts dairy product processing wastewater from high-concentration wastewater produced during a production process of a dairy product enterprise in Shaanxi Province. It is tested that the BOD₅ of the wastewater is 55,340 mg/L.

### Example 1

A method for cultivation of black soldier flies using high-concentration organic wastewater, mainly including the following steps:
(1) collecting meal waste, removing impurities such as plastic and metal that cannot be eaten by the black soldier flies, pulverizing the waste into 10-mesh particles using a pulverizer, and then separating and collecting meal wastewater using a three-phase separation device;
(2) adding the meal wastewater into dry peanut meal to achieve a water content of peanut meal of 65%, taking yeast (purchased from the China Center of Industrial Culture Collection, with the Chinese name Yarrowia lipolytica, and the No. CICC31829) which is 0.6% of a mass of the dry peanut meal, and fermenting at a temperature of 35° C. for 7 days;
(3) adding modified zeolite, folic acid, and choline chloride into a fermentation product, stirring uniformly, where a mixture includes 700 parts of fermentation product, 28 parts of modified zeolite, 0.0003 parts of folic acid, and 0.16 parts of choline chloride; and adding the meal wastewater to adjust a humidity of the mixture to 75% to obtain a feed; and
(4) feeding 6-day-old BSFLwith the resulting feed, where water evaporation and consumption occur in a feeding process, and the meal wastewater is added daily to keep a humidity of the feed at 75%.

### Example 2

A method for cultivation of BSFL using high-concentration organic wastewater, mainly including the following steps:
(1) collecting meal waste, removing impurities such as plastic and metal that cannot be eaten by the black soldier flies, pulverizing the waste into 10-mesh particles using a pulverizer, and then separating and collecting meal wastewater using a three-phase separation device;
(2) adding the meal wastewater into dry peanut meal to achieve a water content of peanut meal of 70%, taking Bacillus subtilis (purchased from the China Center of Industrial Culture Collection, with the Chinese name Bacillus subtilis, and the No. CICC10446) which is 0.5% of a mass of the dry peanut meal, and fermenting at a temperature of 35° C. for 7 days;
(3) adding modified zeolite, folic acid, and choline chloride into a fermentation product, stirring uniformly, adding the meal wastewater to adjust a humidity of a mixture to 80% to obtain a feed, where the resulting feed includes 600 parts of fermentation product, 22 parts of modified zeolite, 0.0001 parts of folic acid, and 0.12 parts of choline chloride; and
(4) feeding 6-day-old BSFL with the resulting feed, where water evaporation and consumption occur in a feeding process, and the meal wastewater is added daily to keep a humidity of the feed at 80%.

### Example 3

A method for cultivation of BSFL using high-concentration organic wastewater, mainly including the following steps:
(1) collecting meal waste, removing impurities such as plastic and metal that cannot be eaten by the BSFL, pulverizing the waste into 10-mesh particles using a pulverizer, and then separating and collecting meal wastewater using a three-phase separation device;
(2) adding the meal wastewater into dry peanut meal to achieve a water content of peanut meal of 75%, taking yeast (purchased from the China Center of Industrial Culture Collection, with the Chinese name Yarrowia lipolytica, and the No. CICC31829) which is 0.8% of a mass of the dry peanut meal, and fermenting at a temperature of 35° C. for 7 days;
(3) adding modified zeolite, folic acid, and choline chloride into a fermentation product, stirring uniformly, adding the meal wastewater to adjust a humidity of a mixture to 70% to obtain a feed, where the resulting feed includes 500 parts of fermentation product, 14 parts of modified zeolite, 0.00005 parts of folic acid, and 0.14 parts of choline chloride; and
(4) feeding 5-day-old BSFL with the resulting feed, where water evaporation and consumption occur in a feeding process, and the meal wastewater is added daily to keep a humidity of the feed at 70%.

### Example 4

A method for cultivation of BSFL using high-concentration organic wastewater, mainly including the following steps:
(1) collecting meal waste, removing impurities such as plastic and metal that cannot be eaten by the black soldier flies, pulverizing the waste into 10-mesh particles using a pulverizer, and then separating and collecting meal wastewater using a three-phase separation device;
(2) adding the meal wastewater into dry peanut meal to achieve a water content of peanut meal of 75%, taking lactic acid bacteria (purchased from the China Center of Industrial Culture Collection, with the Chinese name Lactobacillus acidophilus, and the No. CICC6082) which is 0.7% of a mass of the dry peanut meal, and fermenting at a temperature of 35° C. for 7 days;
(3) adding modified zeolite, folic acid, and choline chloride into a fermentation product, stirring uniformly, adding the meal wastewater to adjust a humidity of a mixture to 75% to obtain a feed, where the resulting feed includes 700 parts of fermentation product, 24 parts of modified zeolite, 0.0002 parts of folic acid, and 0.15 parts of choline chloride; and
(4) feeding 5-day-old BSFL with the resulting feed, where water evaporation and consumption occur in a feeding process, and the meal wastewater is added daily to keep a humidity of the feed at 75%.

### Example 5

A method for cultivation of BSFL using high-concentration organic wastewater, mainly including the following steps:
(1) collecting dairy product processing wastewater; adding the dairy product processing wastewater into dry peanut meal to achieve a water content of peanut meal of 65%, taking yeast (purchased from the China Center of Industrial Culture Collection, with the Chinese name Yarrowia lipolytica, and the No. CICC31829) which is 0.6% of a mass of the dry peanut meal, and fermenting at a temperature of 35° C. for 7 days;
(2) adding modified zeolite, folic acid, and choline chloride into a fermentation product, stirring uniformly, where a mixture includes 700 parts of fermentation product, 28 parts of modified zeolite, 0.0003 parts of folic acid, and 0.16 parts of choline chloride; and adding the dairy product processing wastewater to adjust a humidity of the mixture to 80% to obtain a feed; and
(3) feeding the 6-day-old BSFL with the resulting feed, where water evaporation and consumption occur in a feeding process, and the dairy product processing wastewater is added daily to keep a humidity of the feed at 80%.

### Comparative Example 1

(1) adding tap water into dry peanut meal to achieve a water content of peanut meal of 65%, taking yeast (purchased from the China Center of Industrial Culture Collection, with the Chinese name Yarrowia lipolytica, and No. CICC31829) which is 0.6% of a mass of the dry peanut meal, and fermenting at a temperature of 35° C. for 7 days;
(2) adding modified zeolite, folic acid, and choline chloride into a fermentation product, stirring uniformly, where a mixture includes 700 parts of fermentation product, 28 parts of modified zeolite, 0.0003 parts of folic acid, and 0.16 parts of choline chloride; and adding the tap water to adjust a humidity of the mixture to 75% to obtain a feed; and
(3) feeding 6-day-old BSFL with the resulting feed, where water evaporation and consumption occur in a feeding process, and the tap water is added daily to keep a humidity of the feed at 75%.

Compared to Example 1, Comparative Example 1 differs in that meal wastewater is replaced with tap water.

### Comparative Example 2

Compared to Example 1, Comparative Example 2 differs in that natural zeolite is added instead of modified zeolite in step (3), and other operations and parameters remain unchanged.

### Comparative Example 3

Compared to Example 1, Comparative Example 3 differs in that no folic acid is added in step (3), and other operations and parameters remain unchanged.

### Comparative Example 4

Compared to Example 1, Comparative Example 4 differs in that choline chloride is replaced with nicotinic acid in step (3), and other operations and parameters remain unchanged.

### Tests on Feeding BSFL and Analysis of Results

Conservation materials (regular wheat bran+soybean meal+tap water) are used as a control group (CK). Healthy 6-day-old Wuhan-strain black soldier flies of similar stature were sorted into 10 groups (see Table 1), with 200 black soldier flies in each group. Each group was fed daily with sufficient leftover feeds to ensure adequate feed, an ambient temperature is maintained at 28° C., and an air relative humidity is 75%. During the cultivation process, the amount of live black soldier flies in pots and the weight of the leftover feeds after feeding for 13 days were recorded, and the body weights of each group of black soldier flies before and after feeding were recorded. Then, the black soldier flies were freeze-dried, pulverized, and uniformly subjected to Soxhlet extraction (n-hexane extraction solution), and the fatty acid content (%) of the black soldier flies was analyzed by a GC-MS assay (see Table 2).

Various indexes are calculated as follows:
(1) total gains of body weights of BSFL (kg)=A₂−A₁,
where A₂ is the total weight of BSFL after feeding, and A₁ is the total weight of BSFL before feeding;
(2) larva survival rate=number of live larvae after the end of the test/number of live larvae at the start of the test*100%;
(3) feed conversion ratio=gain of weights of larvae/(total adding amount of feed-leftover feed)*100%.

The test results for all examples and comparative examples are shown in Table 1.

TABLE 1

| | Weight | | | |
|---|---|---|---|---|
| | gain of | Feed conversion | Survival | Fatty acid |
| | larva body | ratio (%) | rate | content |
| Group | (g) | (%) | (%) | (%) |
| CKgroup | 32.47 | 16.75 | 96.5 | 85.42 |
| Example 1 | 37.13 | 21.79 | 95.5 | 95.74 |
| Example 2 | 36.70 | 21.34 | 95.5 | 96.28 |
| Example 3 | 36.18 | 20.95 | 95.0 | 95.07 |
| Example 4 | 35.76 | 20.21 | 95.0 | 95.36 |
| Example 5 | 36.33 | 21.42 | 95.5 | 95.52 |
| Comparative | 29.83 | 14.33 | 94.0 | 87.61 |
| Example 1 | | | | |
| Comparative | 25.74 | 9.58 | 69.5 | 70.35 |
| Example 2 | | | | |
| Comparative | 31.46 | 18.05 | 91.5 | 84.50 |
| Example 3 | | | | |
| Comparative | 30.22 | 17.98 | 89.0 | 80.19 |
| Example 4 | | | | |

TABLE 2
Comparison of Some Fatty Acid Components and Contents (%)

| | Example | Example | Comparative | Comparative | Comparative | Comparative | CK |
|---|---|---|---|---|---|---|---|
| | 1 | 5 | Example 1 | Example 2 | Example 3 | Example 4 | group |
| Stearic acid | 2.57 | 2.79 | 2.36 | 2.15 | 1.79 | 1.84 | 4.08 |
| Lauric acid | 34.43 | 33.82 | 23.55 | 26.06 | 22.81 | 14.59 | 11.81 |
| Pentadecanoic | 0.18 | 0.21 | 0.14 | 0.11 | 0.17 | 0.24 | 0.074 |
| acid | | | | | | | |
| Myristic | 5.89 | 5.16 | 5.73 | 4.35 | 5.24 | 6.05 | 0.26 |
| acid | | | | | | | |
| Oleic acid | 12.38 | 13.74 | 11.75 | 10.48 | 11.33 | 10.59 | 24.15 |
| Palmitic | 14.43 | 13.45 | 15.98 | 15.25 | 13.12 | 16.94 | 18.46 |
| acid | | | | | | | |
| Linoleic | 17.85 | 18.40 | 17.04 | 18.67 | 18.28 | 24.65 | 20.39 |
| acid | | | | | | | |

### Analysis of Results

In combination with the data shown in Table 1 and Table 2, the feeding of BSFL with the feed prepared in accordance with the examples of the present disclosure provides significant advantages in terms of not only increased growth performance such as body weight gains, survival rates, and feed conversion ratios, but also improved fatty acid compositions and contents of BSFL. Furthermore, the present study shows that the fatty acid compositions of BSFL are changed due to their feed base stocks and feeding conditions compared to control group, particularly the compositions of lipid materials or protein, and carbohydrate in the feed are different. The present disclosure has an excellent effect in increasing the fatty acid content of larva bodies by regulating their fatty acid compositions by adjusting feed formulations and feeding conditions.

Compared to Example 1, tap water was used instead of meal wastewater for the feed configuration and the addition for feeding in Comparative Example 1, the weight gains and the feed conversion ratios of the BSFL fed were significantly lower than those of Example 1. Most importantly, there is no significant difference in larva survival rate after changing to tap water, suggesting that in the technical solution of the present disclosure, the black soldier flies can recycle toxic and harmful substances in the meal wastewater through intra-abdominal conversion, and the meal wastewater added in bulk does not negatively affect the survival rate of the BSFL. In addition, a large amount of fats, proteins, and the like in the meal wastewater increase the body weight gains, feed conversion ratios, and body fatty acid contents of black soldier flies compared to tap water. The survival rate, body weight gain, feed conversion ratio, and fatty acid content of black soldier flies were all significantly decreased in Comparative Example 2, which may be due to the fact that the modified zeolite in the examples can specifically adsorb harmful substances in the meal wastewater and promote feeding of the BSFL, thereby improving the survival rate, body weight gain, and feed conversion ratio of the BSFL and promoting fatty acid conversion. In the case where no folic acid was added in Comparative Example 3, the fatty acid content of larvae was significantly decreased, indicating that the folic acid can promote the conversion of fatty acids, and at the same time, improve the feeding and conversion of BSFL to the feed prepared by recycling waste the meal wastewater according to the present disclosure. Niacin is used in combination with folic acid in Comparative Example 4, resulting in a decrease in fatty acid content of larvae, indicating that the co-action of folic acid and choline chloride promotes the conversion of fatty acids by black soldier flies, especially, the difference in lauric acid content is significant, it is possible that lauric acid has the function of enhancing immunity of larvae, protecting larvae from lipid oxidation, and making black soldier flies to have stronger tolerance to the environment with high-concentration organic wastewater.

It is shown by the above results that the feeding of BSFL with the feed provided by the present disclosure has good effects, and the present disclosure replaces tap water with high-concentration COD or BOD₅ organic wastewater, prepares the feed with mixing multiple times, and does not perform industrial treatment such as coagulation on organic wastewater, thereby not only realizing recycling of high-concentration organic wastewater, but also improving growth performance and fatty acid contents of black soldier flies, reducing farming costs, and improving economic efficiency.

TABLE 3

| | Example | Example | | | Example | |
|---|---|---|---|---|---|---|
| Item | 1 | 2 | Example 3 | Example 4 | 5 | CK group |
| Crude | 28.57 | 28.83 | 27.65 | 26.49 | 27.03 | 22.84 |
| protein % | | | | | | |
| Crude ash % | 46.82 | 45.12 | 47.45 | 47.82 | 46.35 | 32.46 |
| pH | 6.83 | 6.95 | 6.87 | 6.80 | 6.96 | 6.67 |
| Organic | 75.80 | 74.56 | 74.98 | 73.53 | 73.85 | 67.41 |
| matter % | | | | | | |
| Total | 4.85 | 4.67 | 4.53 | 4.39 | 4.61 | 3.74 |
| nitrogen % | | | | | | |
| Total | 1.79 | 1.87 | 1.71 | 1.68 | 1.52 | 1.26 |
| phosphorus % | | | | | | |
| Total | 0.84 | 0.77 | 0.69 | 0.72 | 0.58 | 0.35 |
| potassium % | | | | | | |

On the other hand, the influence of high-concentration organic wastewater on the nutrient content of insect droppings is shown in Table 3, after farming black soldier flies using the feed prepared in the present disclosure for 15-20 days, the water content of insect droppings produced is controlled at around 40%-50%, and separation of insect bodies and insect droppings is facilitated; and after the insects are collected, the remaining insect droppings are then fermented to produce animal feeds or raw materials having high proteins and organic acids after trans-abdominal predigestion of black soldier flies. It can be used for producing chicken feeds, swine feeds, aquatic feeds, and herbivorous feeds. That is, the present disclosure can also achieve direct feedification of insect droppings.

Example 1: Test for cultivation of laying hens by feedification of insect litter of black soldier flies:

### (1) Test Materials and Test Methods

### 1 Test Materials

### 1.1 Test Animals

360 40-week-old healthy Jinghong No. 1 laying hens with similar weights and egg-laying rates were randomly divided into 3 treatment groups, with 6 replicates in each treatment group and 20 laying hens in each replicate. Fully closed environmental control cage-rearing chicken houses were used, with 4 laying hens housed per metal cage; and during the test, the laying hens were allowed to freely eat and drink water, the droppings were removed daily, regular disinfection is performed, and the houses were kept well-ventilated, clean, dry and hygienic. The formal test was started after 1 week of pre-feeding, and the formal test period was 28 days.

The control group was fed with a corn meal and soybean meal type basal ration (see Table 4); and the peanut meal group and the insect litter group were that peanut meal or peanut meal fermented insect litter (based on step (4) of Example 1: insect litter obtained after feeding 6-day-old larvae of black soldier flies with the resulting feed) were added respectively for replacing 30% of soybean meal on the basis of basal ration. The feed adding amount, death and culling rate, egg laying rate, and egg weight were recorded daily, the feed intake was counted weekly, and the production performance and egg quality of the laying hens were recorded, observed, and determined.

### 1.2 Main Instrument Equipment

EFR-01 Egg Shell Strength Tester, ETG-1061 Egg Shell Thickness Tester, EA-01 Multifunctional Egg Quality Analyzer, BSA2201-CW Electronic Precision Balance, MA160-1CN Infrared Moisture Tester, Benchtop High-Speed Freeze Centrifuge, DW-86L626 Ultra-Low-Temperature Refrigerator, DHG-9145A Air-Blasting Drying Box, HH-2 Numerical Display Constant Temperature Water Bath, BF-10 Small High-Speed Pulverizer, and SCIENTZ-50YG/A Freeze Dryer.

TABLE 4
Nutrient Standard Composition of Basic Ration

| | Addition Proportion |
|---|---|
| Item | (%) |
| Corn | 61.02 |
| Soybean meal | 23.17 |
| Soybean oil | 1.50 |
| Wheat bran | 3.77 |
| Limestone | 8.50 |
| Calcium hydrogen | 1.12 |
| phosphate | |
| Sodium chloride | 0.28 |
| L-lysine sulfate | 0.15 |
| DL-methionine | 0.09 |
| Premix | 0.40 |
| Total | 100.00 |

### 2. Experiment Method

### 2.1 Determination of Production Performance

Average daily feed intake: The feed intake for each group was recorded weekly, and the average daily feed intake was calculated. Average daily feed intake=total feed intake/number of days/stock of laying hens.

Average egg weight: The total egg weight of each group was recorded daily, and the average daily egg weight was calculated. Average daily egg weight=total daily egg weight/daily number of eggs laid.

Average egg laying rate: The number of eggs laid and the stock of laying hens in each group were recorded daily, and the average daily egg laying rate was calculated. Daily egg laying rate=number of eggs laid daily/stock of laying hens.

Feed-gain ratio: The total egg weight and consumed amount of each group were recorded daily, and the feed-gain ratio of each group was calculated. Feed-gain ratio=consumed amount/total egg weight.

Death and culling rate: The number of laying hens dead and culled in each group was recorded daily, and the death and culling rate for each group is calculated. Death and culling rate=number of laying hens dead and culled/number of laying hens entering the house.

### 2.2 Determination of Egg Quality

The egg shell strength was determined using the EFR-01 Egg Shell Strength Meter, with eggs placed vertically on the meter with blunt end up and the unit set at kg/cm2 during the determination.

The egg shell thickness was measured using the ETG-1061 Egg Shell Thickness Meter, the thicknesses of the tip, middle and blunt end of the egg were measured, and the average thickness of the egg shell was calculated.

The egg weight, yolk color, albumen height and Haugh unit were determined using the EA-01 Multifunctional Egg Quality Analyzer, and after the eggs were knocked down, the fresh eggs were placed in the center of the tray of the analyzer.

### (2) Test Results and Analysis

1 Effect of peanut meal and fermented insect litter on production performance of laying hens

TABLE 5
Production Performance

| | Control | Peanut | Insect |
|---|---|---|---|
| Item | group | meal group | litter group |
| Average daily feed | 114.64 | 113.87 | 116.18 |
| intake (g) | | | |
| Average egg weight | 61.76 | 60.23 | 61.68 |
| (g) | | | |
| Average egg laying | 83.14 | 82.33 | 83.56 |
| rate (%) | | | |
| Feed-egg ratio | 2.23 | 2.30 | 2.25 |
| Death and culling rate | 3.89 | 5.01 | 3.27 |
| (%) | | | |

The test results were shown in Table 5. Compared to the control group, the peanut meal group was adversely affected in terms of the feed intake, death and culling rate, and other indexes due to the influence of miscellaneous meal, while the peanut meal fermented insect litter increased the average daily feed intake of the laying hens, decreased the death and culling rate, and had no significant effect on the average egg weight and average egg laying rate. The above results show that the peanut meal fermented insect litter can improve the reduction in production performance caused by feeding peanut meal to laying hens and reduce the death and culling rate.

Effect of peanut meal and fermented insect litter on egg quality of laying hens

TABLE 6
Egg Quality

| | Control | Peanut | Insect |
|---|---|---|---|
| Item | group | meal group | litter group |
| Egg weight (g) | 61.79 | 60.72 | 61.64 |
| Eggshell strength | 3.55 | 3.67 | 3.69 |
| (kg/cm²) | | | |
| Eggshell thickness | 0.37 | 0.39 | 0.39 |
| (mm) | | | |
| Color of egg yolk | 7.82 | 8.01 | 7.99 |
| Protein height (mm) | 6.83 | 6.92 | 7.12 |
| Haugh unit | 82.03 | 82.95 | 83.94 |

Test results were shown in Table 6. Compared to the control group, the peanut meal fermented insect litter increased the egg shell strength, yolk color, albumen height, and Haugh unit of laying hens, and had no significant effect on the egg weight and egg shell thickness. The above results show that the peanut meal fermented insect litter can increase the egg shell quality and the freshness of the eggs.

Example 2: Test for cultivation of broiler chickens by feedification of black soldier fly insect litter:

### (1) Test Materials and Test Methods

### 1 Test Materials

### 1.1 Test Animals

360 1-day-old healthy Hubbard broiler chickens were randomly divided into 3 treatment groups, with 6 replicates in each treatment group and 20 broiler chickens in each replicate. Fully closed environmental control cage-rearing chicken houses were used, with 4 broiler chickens housed per metal cage; and during the test, the broiler chickens were allowed to freely eat and drink water, the droppings were removed daily, regular disinfection is performed, and the houses were kept well-ventilated, clean, dry and hygienic. The test period was 42 days. The control group was fed with a corn meal and soybean meal type basal ration (see compositions in Table 7), treatment is performed as Example 1, and peanut meal or peanut meal fermented insect litter were added respectively for replacing 30% of soybean meal to obtain the peanut meal group and the insect litter group. The loading amount and death and culling rate were recorded daily, the feed intake and body weight were counted weekly, and production performance and slaughter performance of broiler chickens were recorded, observed, and determined.

### 1.2 Main Instrument Equipment

BSA2201-CW Electronic Precision Balance, MA160-1CN Infrared Moisture Tester, Benchtop High-Speed Freeze Centrifuge, DW-86L626 Ultra-Low-Temperature Refrigerator, DHG-9145A Air-Blasting Drying Box, HH-2 Numerical Display Constant Temperature Water Bath, BF-10 Small High-Speed Pulverizer, and SCIENTZ-50YG/A Freeze Dryer.

TABLE 7
Composition of Basic Ration

| | Addition |
|---|---|
| Item | Proportion (%) |
| Corn | 61.48 |
| Soybean meal | 23.67 |
| Soybean oil | 1.85 |
| Corn gluten meal | 4.36 |
| Wheat bran | 3.43 |
| Limestone | 1.45 |
| Sodium chloride | 0.35 |
| L-lysine sulfate | 0.53 |
| DL-methionine | 0.35 |
| L-threonine | 0.25 |
| Isoleucine | 0.18 |
| Calcium hydrogen | 1.62 |
| phosphate | |
| Choline chloride | 0.08 |
| Premix | 0.40 |
| Total | 100.00 |

### 2. Experiment Method

### 2.1 Determination of Production Performance

Average daily feed intake: The feed intake for each group was recorded weekly, and the average daily feed intake was calculated. Average daily feed intake=total feed intake/number of days/stock of broiler chickens.

Average daily weight gain: The body weight of each group was recorded weekly, and the average daily weight gain was calculated. Average daily gain=(final weight-initial weight)/days of the test.

Feed-weight ratio: The weight gain and consumed amount of each group were recorded weekly, and the feed-weight ratio of each group was calculated. Feed-weight ratio=average daily feed intake/average daily weight gain.

Death and culling rate: The number of broiler chickens dead and culled in each group was recorded daily, and the death and culling rate for each group is calculated. Death and culling rate=number of broiler chickens dead and culled/number of broiler chickens entering the house.

### 2.2 Determination of Slaughter Performance

At the end of the test, 10 broiler chickens were randomly selected from each group to be slaughtered, and the slaughter percentage, the half-gutting rate and the full-gutting rate were calculated. Live weight: Body weight of broiler chickens fasted 12 h before slaughter. Carcass weight: Body weight after removal of feathers, foot cuticle and toe shell after bloodletting. Half-gutting weight: carcass weight after removal of trachea, esophagus, spleen, pancreas, biliary, crop, reproductive organs and gut. Full-gutting weight: Body weight obtained by removing the heart, liver, kidney, myostomach, glandulostomach, fat, head, and feet from the half-gutting weight.
Slaughter percentage=carcass weight/live weight
Half-gutting rate=half-gutting weight/live weight
Full-gutting rate=full-gutting weight/live Weight

### (2) Test Results and Analysis

1 Effect of peanut meal and fermented insect litter on production performance of broiler chickens

TABLE 8
Production Performance

| | Control | Peanut | Insect |
|---|---|---|---|
| Item | group | meal group | litter group |
| Average daily feed | 108.89 | 108.04 | 108.72 |
| intake (g) | | | |
| Average daily weight | 65.82 | 64.89 | 65.72 |
| gain (g) | | | |
| Feed-weight ratio (g) | 1.65 | 1.67 | 1.65 |
| Death and culling rate | 3.45 | 3.53 | 3.14 |
| (%) | | | |

Test results were shown in Table 8. Compared to the control group, the peanut meal fermented insect litter reduced the death and culling rate of broiler chickens, and had no significant effect on average daily feed intake and average daily weight gain. The above results show that the peanut meal fermented insect litter can improve the reduction in production performance caused by feeding peanut meal to broiler chickens and reduce the death and culling rate.

2 Effect of peanut meal and fermented insect litter on slaughter performance of broiler chickens

TABLE 9
Slaughter Performance

| | Control | Peanut | Insect |
|---|---|---|---|
| Item | group | meal group | litter group |
| Slaughter rate (%) | 88.70 | 87.38 | 90.36 |
| Half-gutting rate (%) | 83.21 | 82.44 | 83.47 |
| Full-gutting rate (%) | 64.85 | 62.97 | 65.03 |

Test results were shown in Table 9. Compared to the control group, the peanut meal fermented insect litter increased the slaughter rate of broiler chickens, and had no significant effect on the half-gutting rate and the full-gutting rate. The above results show that, in comparison with three sets of tests, the peanut meal fermented insect litter can improve the slaughter performance reduction caused by feeding of broiler chickens with peanut meal.

Obviously, the above embodiments of the present disclosure are merely examples made for clearly illustrating the technical solutions of the present disclosure, and are not intended to limit the specific embodiments of the present disclosure. Any modifications, equivalent replacements, and improvements made within the spirit and principle of the claims of the present disclosure shall fall within the scope of protection of the claims of the present disclosure.

What is claimed is:

1. A method for cultivation of black soldier fly larvae using high-concentration organic wastewater, comprising the following steps:
S1. collecting organic wastewater, adding the organic wastewater into plant-based feed to achieve a moisture content of 60% or above, and inoculating a fermentation agent for fermentation treatment to obtain a fermentation product; then, adding modified zeolite, folic acid and choline chloride into the fermentation product, and stirring uniformly to obtain a feed; and
S2. feeding the black soldier fly larvae with the feed obtained in step S1, and maintaining a humidity of the feed by directly supplementing the organic wastewater of step S1 according to a water evaporation condition and content in a feeding process.

2. The method for cultivation of black soldier fly larvae using high-concentration organic wastewater according to claim 1, wherein COD or BOD₅ in the organic wastewater in step S1 is greater than 50,000 mg/L.

3. The method for cultivation of black soldier fly larvae using high-concentration organic wastewater according to claim 1, wherein the organic wastewater in step S1 is selected from one or a mixture of more of meal wastewater, dairy product processing wastewater, fruit processing wastewater, soybean processing wastewater, brewing wastewater, starch saccharification wastewater, and amino acid and organic acid fermentation wastewater.

4. The method for cultivation of black soldier fly larvae using high-concentration organic wastewater according to claim 1, wherein an amount of the organic wastewater added in step S1 enables a water content of the plant-based feed to reach 65%-85%.

5. The method for cultivation of black soldier fly larvae using high-concentration organic wastewater according to claim 1, wherein the plant-based feed in step S1 is selected from soybean meal, rapeseed meal, cottonseed meal, peanut meal, sunflower meal, palm kernel meal, coconut meal, dry and wet distillers' grains, DDGS, Chinese medicine residues, and other plant-based raw materials rich in fiber and protein.

6. The method for cultivation of black soldier fly larvae using high-concentration organic wastewater according to claim 1, wherein the modified zeolite in step S1 is obtained by a modification method of reducing a silica-alumina ratio, KCl and NaOH are dissolved in water, Al(OH)₃ is dissolved in a hot KCl and NaOH solution, natural zeolite is added into the solution and stirred at a normal temperature for 3-5 h, the solution is separated, water is used for washing until neutral, and after evaporation, a solid is calcined at 350-500° C. for 1.5-2 h.

7. The method for cultivation of black soldier fly larvae using high-concentration organic wastewater according to claim 1, wherein the fermentation agent in step S1 is one or a mixture of more of yeast, Bacillus subtilis, and lactic acid bacteria.

8. The method for cultivation of black soldier fly larvae using high-concentration organic wastewater according to claim 1, wherein a using amount of the fermentation agent in step S1 is 0.5%-0.8% of a mass of the plant-based feed.

9. The method for cultivation of black soldier fly larvae using high-concentration organic wastewater according to claim 1, wherein the feed in step S1 comprises 500-700 parts of fermentation products, 14-28 parts of modified zeolite, 0.00005-0.0003 parts of folic acid, and 0.12-0.16 parts of choline chloride, and the organic wastewater is added to adjust the humidity of the feed at 60%-80%.

10. Use of the method according to claim 1 in improving growth performance of black soldier fly larvae and a larva body fatty acid content and insect litter obtained by the method in ameliorating adverse effects caused by use of miscellaneous meal in livestock and poultry farming processes.
